(19) United States
(12) Patent Application Publication
(10) Pub. No.: US 2017/0146460 A1
(43) Pub. Date: May 25, 2017

(54) CONTROLLED ENVIRONMENT OBSERVATION DEVICE

(71) Applicants: Michael D. Cecchi, Longboat Key, FL (US); Monica Mezezi, Guelph (CA); Michael Ryan Cecchi, Longboat Key, FL (US); Aami Mezezi, North Redington (FL)

(72) Inventors: Michael D. Cecchi, Longboat Key, FL (US); Monica Mezezi, Guelph (CA); Michael Ryan Cecchi, Longboat Key, FL (US); Aami Mezezi, North Redington (FL)

(21) Appl. No.: 15/352,195

(22) Filed: Nov. 15, 2016

Related U.S. Application Data
(60) Provisional application No. 62/258,154, filed on Nov. 20, 2015.

(51) Int. Cl. G01N 21/84; C12Q 1/02

(52) U.S. Cl. CPC G01N 21/84; C12Q 1/02

(57) ABSTRACT

A controlled environment observation device comprising: a body; a heated shelf located in the body; a plurality of shelf openings in the heated shelf; a plurality of embryonic chambers removably installed in the plurality of shelf openings, each of the plurality of embryonic chambers comprising: an optically transparent heated shelf; a specimen dish removably attached to the top of the optically transparent heated shelf, the specimen dish having a generally optically transparent bottom; an x,y,z camera system located in each of the embryonic chambers and below the specimen dish and the optically transparent heated shelf; a computer located in the controlled environment observation device; and a controller in signal communication with the xyz camera system and the computer. A controlled environment observation device comprising: a body; at least one multi-area embryonic chamber located in the body, the multi-area embryonic chamber comprising: at least one outer wall forming a perimeter; a platform generally located within the perimeter; an opening in the platform; an optically transparent heated shelf located in the opening; a first plurality of inner walls forming a second plurality of volumes with the at least one outer wall; a second plurality of specimen dishes configured to be removeably attached to the optically transparent heated shelf in each of the second plurality of volumes, each of the specimen dishes having a generally optically transparent bottom; an x,y,z camera system located beneath the optically transparent heated shelf, and configured to move a camera to any of the specimen dishes.

### CROSS-REFERENCES

This patent application claims the benefit of provisional patent application No. 62/258,154; by Michael D. Cecchi, Monica Mezezi, Michael Ryan Cecchi, and Aami Mezezi, entitled “CONTROLLED ENVIRONMENT OBSERVATION DEVICE”, filed on Nov. 20, 2015, and which provisional application is fully incorporated by reference herein.

### TECHNICAL FIELD

This invention relates to a device which provides for a controlled environment for the culturing and observation of biological specimens including embryos, oocytes and stem cells.

### BACKGROUND

Currently, there are several known microscope type devices, which provide a means for observing biological sample. These known devices have a limited ability to take good images without the use of a manual-focusing device. These devices lack the ability to take highly accurate and repeatable pictures of embryos and send this information to a user. A problem with the current devices is that they mostly utilize manual focusing systems and which often takes a great deal of time to achieve a good focal imaging and is not readily repeatable.

An additional problem with current devices is that the components are often separate and not attached in such a fashion as to have the components a more exacting distance from each other. This may lead to the lense distance from the observation platform to vary and therefore doses not give the instrument the best repeatable images distance.

An additional problem facing current devices is that they may require several different magnification powers of the lenses in order to achieve the magnification of the images desired.

Some of the additional problems facing current devices are that they may not be able to move the imaging device with respect to the biological specimen to obtain proper images of the specimen. Therefore the dish or holding device must be manually moved about to find the specimen and obtain a proper and satisfactory image.

Another problem faced by current devices is that they are unable to obtain proper images for specimens that may roll or otherwise move.

Some current devices do not provide all the components necessary to give the embryos the best chance to survive and be the best for preimplantation. Some of the current devices are cumbersome, have a large amount of service requirements and are not consistent in their performance.

An additional problem faced by some current devices is that they have an inferior or inadequate filtering system to provide continual clean air for the biological specimen environment. The current devices do not have consistent methods for monitoring the Volatile Organic Compounds (VOC) and inform users on the timely replacing of the filters. The current devices may also lack the filter technology necessary to keep the air quality acceptable in the biological environment

Some current devices are cumbersome and heavy, and they have separate systems for the incubator environment and camera systems, which make the service and repair difficult.

Current devices often use flat bottom dishes or devices, which allow the embryos to be anywhere on the bottom of the dish, making imaging more difficult, does not allow flexibility in the magnification of the images, will allow the embryo to rest on the edges of the dish, which may distort its shape and appearance and does not allow the use of a large quantity of media, due to the limited well size of the dishes. Although a limited well size makes finding the embryo easier, smaller amounts of media will impede embryo development and may not allow the embryo to grow and culture properly.

A major problem of some current devices, is that they may have a ‘large box’ or larger holding stations and equipment configuration. These large box devices are typically assembled in the traditional manner of multiple parts being added to the ‘large box’ device. This creates difficulty in servicing the system due to the many parts in the ‘large box’ device, and the lack of modularity, such that one component cannot easily be replaced. Thus large box devices typically are very complicated to provide service and repair.

Another major problem of current devices is that they often comprise many moving parts, which need to work together in the field, but are difficult to service and repair.

Many current devices require an on-site service call by a qualified service repair person for any and all mechanical failures of the products. These disadvantages become more critical when considering the fact that the parts needed to repair the product may not be readily available for the immediate service needs and may need to be placed on order, shipped and then serviced, resulting in the unit being inoperative for an extended period of time, causing possible damage or death to the biological specimen.

Another major problem facing the current devices is that they typically use one camera and one mechanical system for multiple biological specimens such as embryos. This means that there may be up to nine different biological specimens in different dishes, or are separated by space within a single device. If the camera or any of the key mechanical systems fail to work properly the entire system cannot be used. This may result in the transportation of the patient embryos in one system to another system, or the user not being able to important features of the device such as taking photographs.

An additional problem of current devices is that the devices generally only have one system for providing incubation to the entire device. If this incubation system malfunctions, such as temperature, gas mixes or power malfunction, the entire unit may become useless as to the incubation function. This may result in loss of embryos, reduced growth of the embryos and the emergency transfer of all patient embryos to a second unit. The problem most clinics will face in this situation is the likelihood, they will not have a backup unit or a second unit with space to accommodate the transferring embryos and therefor the user must use a conventional incubator system, without the camera features and therefore not be able to take the desired images. The failure of the incubator system may result more significant problems such as the loss of embryos.

An additional disadvantage to the current devices is that they are generally built to accommodate a defined number of dishes and therefore limit the number of biological specimens that may be serviced at the same time in the unit.

A problem facing the other types of incubator systems, is that they may use indirect heating, which may not provide appropriate heating to the embryo environment.

Many of the existing systems do not contain any safeguards to keep unauthorized personnel from entering the software system and/or the hardware systems of the devices. Most existing systems do not restrict individuals from entering the software system and from entering the chambers or incubator portion of the device, which houses the embryos. In addition there also do not appear to be systems which will keep unauthorized personnel from making entries to the software.

In many existing systems, the user will use an older or lightly used standard incubator, which may be available, to pre-equilibrate dishes, media and oils. In most cases these already existing systems run on its own system of electricity, and gases. These electrical and gas system generally must be maintained separately.

Many of the products on the market today do not contain the feature of having separate chambers. Many products have a system where the dishes share the environment, gases and camera system.

The ability to take quality, timely images of the embryos has been a problem facing all the current systems. The other system encounters a problem with aligning with the exact location of the embryo when it is time to take an image.

Currently, most systems utilize a single dish or holding configuration for holding the embryos during the process.

Thus there is a need for a controlled environment observation device that overcomes the above listed and other disadvantages.

### SUMMARY OF THE INVENTION

The invention relates to a controlled environment observation device comprising: a body; a heated shelf located in the body; a plurality of shelf openings in the heated shelf; a plurality of embryonic chambers removably installed in the plurality of shelf openings, each of the plurality of embryonic chambers comprising: an optically transparent heated shelf; a specimen dish removably attached to the top of the optically transparent heated shelf, the specimen dish having a generally optically transparent bottom; an x,y,z camera system located in each of the embryonic chambers and below the specimen dish and the optically transparent heated shelf; a computer located in the controlled environment observation device; and a controller in signal communication with the xyz camera system and the computer.

The invention also relates to a controlled environment observation device comprising: a body; at least one multi-area embryonic chamber located in the body, the multi-area embryonic chamber comprising: at least one outer wall forming a perimeter; a platform generally located within the perimeter; an opening in the platform; an optically transparent heated shelf located in the opening; a first plurality of inner walls forming a second plurality of volumes with the at least one outer wall; a second plurality of specimen dishes configured to be removeably attached to the optically transparent heated shelf in each of the second plurality of volumes, each of the specimen dishes having a generally optically transparent bottom; an x,y,z camera system located beneath the optically transparent heated shelf, and configured to move a camera to any of the specimen dishes.

### BRIEF DESCRIPTION OF THE DRAWINGS

The present disclosure will be better understood by those skilled in the pertinent art by referencing the accompanying drawings, where like elements are numbered alike in the several figures, in which:

FIG. 1 is a perspective view of the controlled environment observation device;

FIG. 2 is a side cross-sectional view of an embryonic chamber;

FIG. 3 is a view of a specimen dish and an xyz camera system;

FIG. 4 is a side cross-sectional view of another embodiment of an embryonic chamber;

FIG. 5 is a view detailing the articulating stage;

FIG. 6 is a view detailing the articulating stage in another configuration;

FIG. 7 is a top perspective view of another embodiment of an embryonic chamber;

FIG. 8 is a bottom perspective view of the embryonic chamber from FIG. 7;

FIG. 9 is a top 1 view of another embodiment of an embryonic chamger;

FIG. 10 is a side cross-sectional view of the embryonic chamger from FIG. 9; and

FIG. 11 is another embodiment of the disclosed system.

### DETAILED DESCRIPTION

The disclosed device may provide a more accurate focusing on and imaging of biological specimens such as embryos, stem cells, and other biological specimens. The disclosed device will include at least one camera. In one embodiment the camera may be located below the biological specimen and may be moved by a mechanical system—an X,Y,Z camera system. The disclosed device may also comprise an onboard microprocessor to connect the components comprising the disclosed device. The onboard microprocessor may allow the invention to be independently operated and will allow the device to be more portable and replaceable.

The disclosed device may comprise an observation platform, a heated viewing surface, a mechanical X, Y and Z system, and a camera configured to view the underside of the observation platform. By being configured in such a fashion, it allows the camera to be located more accurately with respect to the specimen.

The disclosed device has a unique advantage in utilizing a concise XYZ system. Because the disclosed device is mounted to view the underside of the observation platform, it allows the Z component to move the camera up and down with respect to the observation platform, i.e. closer or further from the specimen.

The disclosed device comprises an incubated environment with the proper levels of CO², N² and oxygen for specimen growth, as well as a camera system, a component to hold culturing specimen dishes in place, the dishes containing a media solution, and an air filtering system for cleaning and recirculating the gases within the system. The disclosed device will include the incubated environment, at least one camera below the dishes which may be moved by a mechanical system, commonly known as an X, Y, Z system, for the accuracy of the images and for the taking of images of the embryos, and an onboard microprocessor to connect the device to equipment for the monitoring and viewing of these images. The onboard microprocessor allows the device to be independently operated and will allow the device to be more portable and replaceable. The specimen dishes will generally have an optically transparent bottom, so that the specimen in the dish can be visible from below the dish.

The disclosed device may be used for placing the specimens in a conventional petri dish or a unique culturing holding device or dish. Placing these dishes in their designated holding areas will allow a camera to take images of the specimens, from underneath at desired time intervals. The dish and the platform may contain a unique keying system to assure that the dish and the specimens within are in the same viewing location on a repeated basis.

The disclosed device may include a camera which may be auto or self-focusing and/or a manually focused camera.

The disclosed device may include an automated locating system for the precise observation and imaging of the desired specimens and a joy stick′ feature, which will allow the user to easily move a camera with respect to a dish or container and locate and observe desired specimens.

The disclosed device may have connectors for incoming gases, electrical input and monitoring and may be constructed to be portable. The disclosed device may be configured to be integratable into a larger system or several systems.

The disclosed device may include probes and sensors to obtain data used to report and validate the performance of the ongoing systems, such as particulates in the atmosphere, monitoring volatile organic compounds (VOC), PH measuring devices, mouse embryo assay testing capabilities and environment evaluations. The disclosed device may maintain acceptable levels of air quality and may provide data to inform the user that conditions are optimal for embryo culture.

The disclosed device may provide the user with images of each embryo, at predetermined time intervals to provide the user with a better understanding of the development of each embryo, to give the user a better idea of which embryos may be a better embryo for implantation, cryopreservation and or such. The disclosed device may provide a complete environment to culture the embryos for a period from about the first day through about blastocyst or about implantation into the patients, which in most cases are about 5 or 6 days. The disclosed device may provide a superior culturing system, along with information, and images that will ultimately allow for better embryo selection, embryo implantation and a better chance of a live birth.

The disclosed device may use various numbers of biological specimen chambers. The disclosed device may be configured to accommodate about 4, about 6, about 8, about 12, and any number of chambers in the main device, and therefore may be able to accommodate more biological specimens at the same time.

An additional advantage of the disclosed device is that the disclosed device may comprise modules, and the modules of the disclosed device may be easily swapped out for repairs and/or replacement.

The disclosed device may contain a location system, a camera system, a controlled environment of temperature and gas levels, along with a uniquely designed dish to hold the embryo in place for imaging. The disclosed device may have the ability to take images at designated and at specific time intervals; and to utilize an embryo culture media which will allow the uninterrupted culturing of the specimen; and an air filtration device for incoming air and recirculate gases, which will provide the enclosed environment with greater reduction of particulate, VOC's and CAC's. The disclosed device may also include a dual air filter system.

The disclosed device may provide a consistent temperature for the biological specimens of about 37 degrees Celsius at all times.

The disclosed device may contain a temperature controlled environment, where the temperature is maintained at about 37 degrees Celsius, and the environment may be humidity free or contain a prescribed humidity. The amount of humidity in the environment may be determined by the impact it may have on the cleanliness and/or fogging of the windows the camera view through and the preference of the users as well as any impact on the biological specimens.

The disclosed device may comprise a power source, incoming gases necessary for the biological specimens, connectors for transmitting images and electronic signal communication with the various components of the disclosed device. The disclosed device may comprise a controlled environment volume, such as chambers or modules. These chambers or modules may maintain a temperature-controlled environment for the biological specimens, with incoming gas ports and a camera system for taking images of the specimens. The disclosed device may comprise gas input ports for the exchange and monitoring of the environmental gases. The disclosed device may include an environmental air filtration and purification system. The disclosed device may use unique combinations of heating, temperature controls, imaging mechanisms, and incoming gas systems, along with certain air flow techniques and HEPA/VOC filters to more effectively clean and maintain a very clean incubated environment for the biological specimens. Images are to be taken from underneath the dish, with a camera system, which may be moved to facilitate the taking of images at specific locations and times.

The disclosed device may also comprise a computer system configured to view and take images of the biological specimens, configured to move the camera, control and display important parameters related to the biological specimens including and not limited to the internal temperature of the disclosed device, internal temperatures of the biological specimen containers, gas levels, such as CO², N² and oxygen.

The disclosed device may comprise a display screen. The display screen may be used to show each patient embryo in a sequence selected by the user. The embryos may be shown at specifically selected time intervals, metabolic stages and at any other times or stages selected by the user. The system will allow the user to compare past and present embryos of the patient, the program and the clinic.

In one embodiment of the disclosed device, the disclosed device may comprise an incubated environment, which may be a generally humidity free or low humidity environment. This environment allows the cameras to take better images of the specimens, because their will be little or no moisture to adversely affect the image taking capability of the disclosed device.

Another embodiment will allow the embryos to be maintained in a humidified environment and the cameras/scope to be outside of that environment and itself in a dry humidity environment. Using a humidified environment will not dry out the embryo's environment. A specially designed specimen dish will allow the embryos to be cultured in both environments, by providing an ample amount of media and will allow for an oil overlay, which has a tendency to keep the media from evaporating and/or helps to maintains a consistent temperature.

An additional embodiment of the disclosed device is that it may be constructed in such a way that the disclosed device may contain many dishes in a single chamber, where the camera moves beneath the dishes and will be able to move to position itself at the exact location where it will take pictures. The dishes and camera may be within the same chamber device.

The disclosed device may have the ability for a specimen chamber to be affixed to an external device, away from the base unit for a period of time, to allow the user to perform various testing procedures or observations. This external device will support the functions of the main unit, during this time.

The disclosed device is has a unique advantage over the competition in that it can incorporate devices and technologies into a far more effective and reliable system for the culturing of embryos. The combination of these devices along with addition of unique key features within this disclosed device, establishing a stand-alone, more complete incubation and maturation device for the purpose of long-term uninterrupted embryo culture.

The disclosed device may contain a unique combination of metals, glass or plastic on the bottom of compartments, where the dish is held. This combination of materials will overcome the problems of maintaining a consistent heat across the bottom of the dish well, and therefore maintain a consistent heat across the bottom of the entire dish, which will result in a constant temperature for the media and oil within the dish and therefore maintain a constant temperature for the embryos to develop in. The ability to maintain a consistent heat across the base of the dish and its content will result in a significant benefit of the disclosed device and will increase the chances of better embryo development and a great success rate.

The dish holding area may be a combination of glass, plastic, metal or a composite, that allows specific areas of the bottom to be clear, allowing the camera to be able to take the images from underneath the dish. The glass or plastic bottom will be integrated with a containment ring or system, which will be replaceable, and changes in and out to allow different dish configurations, changeable if the surface gets scratched. This containment system will insure that the dish aligns with the camera each time to allow imaging. The bottom will contain a combination of the glass and or plastic and a portion of a metal material around and/or across the bottom of the dish holding area.

The metal and composite material may contain a crossing pattern on the bottom of the holding area which will assist in applying a consistent heat in the bottom component, which will maintain a consistent heat across the bottom of the dish, and transfer this heat to the dish, media and oils within the dish.

The disclosed device may include an interchange dish holding component. The interchange dish holding component may be designed to be inserted into the incubator assembly and to be removed and changed to match a particular dish′ heating requirements or when the glass or plastic, of which the images are taken through may become scratched or damaged. The component will be able to meet the heating requirements and the dish configurations to provide the most effective heating available and to meet the needs of the specimens. The bottom of the holding component may become scratched or discolored and may be in need of changing. The interchange dish holding component will allow the disclosed device to be more versatile for the future developments of dishes, material and the needs of the users.

The embryos need to be safely contained against entry by unauthorized personnel. Thus, the disclosed device may contain a combination of security system such as a fingerprint recognition system, key system or keypad entry systems. The recognition system will be used to only allow authorized personnel for access to the software, make changes and the entry into the chambers holding the embryos.

The chambers or the module may be locked closed until such time as an authorized user unlocks the chambers or module. The locking mechanism may be of a mechanical nature, may be a magnetic locking system, may be an electronic locking system, or any suitable locking system currently available depending on the individual requirements of the uses. The locking system will contain safeguards allowing an override of the locking system in case there is a problem with the locking system—such as a loss of electrical power or mechanical failures.

The chamber or module may be used individually or in combination with other chambers; in a larger holding device which will hold 6, 9 or 12 chambers. Each chamber may be an individual chamber or module enclosure to hold the embryos for growth and maturation. The chamber will contain a separate embryo holding component within which will hold the embryos, maintain an accurate and consistent heat for the embryos, gas input channels and the ability to view and take images of the embryos from below.

The chamber is designed to segregate the embryos and to maintain an optimum environment to maintain the proper embryonic development for each embryo while allowing images to be taken of the embryos over an extended period of time, in order to choose the most likely embryos to implant and then result in a live birth.

An additional advantage of the disclosed device are and the separate chambers, which allows the user to remove from the disclosed device a device a single dish from a single chamber, to perform other types of testing such as pre-implantation genetic diagnosis (PGD), intra-cytoplasmic sperm injection (ICSI) or any additional types of analysis or testing, without impacting any of the other chambers, or the dishes, and/or samples within. This feature will additionally allow the removal of a single dish without impacting the environment of the other chambers.

The dishes used, may be those designed to allow the embryo to settle at the bottom, middle of the wells, within the dish. This allows this disclosed device to include a more compact system for the cameras to locate the embryo for imaging. The fact that the embryo rests in the bottom middle of the dish keeps the embryos from settling against the well walls, which may distort the embryo, may not allow adequate viewing of the embryo to allow the finding such key features, such as the embryos spindle.

An additional significant advantage of these unique dish holding areas and the dishes used is that they contain a center and the imaging focal point for the camera to focus on and find easily, but also allow for better-magnified images, and the use of more and greater levels of magnification. Imaging can be taken through the bottom of the dish.

The camera system may have the ability to articulate in order to take images at multiple angles to capture 3D image information, which will be assembled by the system to produce a 3D image for the end user. The 3D imaging will allow the end-user more information and allow them to locate and monitor additional features of the embryos, which may not be visible when taken in a single angle, such as cell development, nuclei and the like.

An additional advantage of the disclosed device is it allows the collection of images at key intervals of development. Imaging will be taken and collected to be used in a determined sequence of images for the evaluation by the user or embryologist. This timed sequencing and maturation sequential will be used to monitor the growth levels and the development of the embryos.

The disclosed device will allow the user to observe and annotate and describing the maturation of a human embryo. This will also allow the comparison of these images to the prior images of same embryos, other embryos of the patient, and embryos from past successful treatments for this clinic or laboratory. The images will be sequenced in a collection of the images in a useful collection for comparisons. These sequences and comparisons of those in times, morphological development and other sequences desired by the user to determine which embryos to be used in implantation or cryopreservation.

The disclosed device will take the images at planned and timely intervals, which may be at intervals of any combination of minutes or hours. The intervals of the images may be adjustable by the user to the frequency they find most effective for their protocols, practice and clinic.

The time intervals and sequences used in the disclosed device will take into account the possible harmful effects that the light used may have on the embryos and their development. Our studies have established the useful light colors and wavelengths for the system, as well as to produce enough light for clear and effective imaging.

The culture media to be used in the disclosed device may be consistent with the media solution used for extended and uninterrupted embryo development. The embryos may be matured from the retrieval, referred to as day “0”; they then may be cultured through to blastocyst development or Day 5 or 6. These embryos have a better chance of implantation and eventual live birth, after they have been matured to the blastocyst stage. This media is consistent with the current media products, trade named Global®, which is a proprietary media solution of the assignee.

The disclosed device will allow the use of a multiple number of medium solutions and protocols.

The disclosed device incorporates a complex system to maintain the temperature of the embryos. The system includes a heating system for the platform for all the chambers and each chamber, as well as temperature reading probes throughout the disclosed device to assure that the temperature are what is need and what is being reported.

The device may include incoming gas ports for the control of the CO², N² and oxygen levels, injector ports and control mechanisms. The disclosed device may include its own gas mixer and sensors for adjusting the gas levels as needed.

The disclosed device is a combination of the described incubator filters and may include at least one motor in the device to provide consistent airflows, air velocities and air exchanges.

The disclosed device may contain a unique system for monitoring the volatile organic compounds (VOC) and particulates levels within the environment on a consistent basis. By reading and monitoring the VOC's, this will allow continual usage of the device while knowing that the environment is correct. Current devices do not include this feature, but rely on the replacing of the air filters in timely basis to insure clean air. These filters may be not working to the full capacity during this time due to loading factors and not having a constant monitoring system.

The disclosed device may include a mechanism to provide a vibration within the device, at planned intervals. This vibration effect has multiple purposes in the process for the improvement of the embryonic development. One accomplishment of the vibration system is to vibrate certain surfaces, the dish or entire device, thereby vibrating the dish. This vibration will allow the consistent mixing of the media solutions, which the embryos are culturing in. The vibration mechanism may create a vibration which can mimic the mother's natural motion, which the embryo would experience through the 5 or 6 days of normal maturation if within the mother's body.

Another importance of the vibration system is to vibrate the dishes and allow the embryos to drift to the bottom of the uniquely designed dishes. This will allow the embryos to be at the center of the dish, at the focal point so that the camera is able to focus on the exact spot the embryo is sitting within the dish for imaging

The disclosed device will have the capacity to provide audio signals and sounds, as well as listen to the any sound within the device. The options may be sounds such as a heartbeat, music, etc.

In addition, the ability to detect sound or motion within the dishes is to alert the system as to any movement by the specimens within the dishes. Embryos as they mature, expand, grow, roll and change shape. The ability to detect such changes would allow the system to alert the camera system, to take key images at embryonic developmental stages. The disclosed device will use this information to coordinate the images with the key morphological changes, such as 2-cell split, 4 cell split, compaction and 8-cell split.

The disclosed device includes a unique feature, which will allow the user to select the dish type they want to use, and the system will provide chooses to recognize the different dish configurations and set up the positioning for the images to be taken for the configuration of each particular dish.

The ability to select the dish type allows the dishes used to be specific for the different needs of the user. To illustrate this embodiment, in one example a dish may contain many smaller wells which may contain fewer amounts of culture media, of approximately 100 microliters of media in the well. A second user may desire to use more media, of up to 300 microliters per well, which would require larger wells, which may position the wells differently in each dish configuration.

The user may at times wish to perform different procedures or protocols and therefore require a different dish and set procedure, which will require different timing intervals, types of images and overall different treatment. The disclosed device will be able quickly adjust to the different dish in the wells and may use a combination of dishes at the same time, in the multiple chambers. This may be accomplished by the user using different modules with different features for the various types of dishes.

The disclosed device may include a separate preliminary chamber to be used to place items into prior to the entry into the main chambers. The use of the preliminary chamber may be to equilibrate media solution prior to use, to obtain the proper pH levels, for the equilibration of pre-prepared dishes and culturing devices, providing a warming feature and media pH equilibration, the temporary holding of specimens prior to the use, and/or to hold certain specimens while the user may be handling a portions of the specimens separately. This preliminary chamber may comprise one chamber, or several chambers. The preliminary chamber(s) may be separate larger chambers than the main chambers used for the embryos.

The preliminary chamber allows the user to place an item into this portion of the disclosed device, whereby it will share the same environmental conditions as the rest of the specimens. This means that specimens will be able to be pre-conditioned to the same balance of gases, temperature and overall environmental conditions. This will likely make the specimens placed into the main chamber more compatible to the environment. The addition of the preliminary chamber (s) also allows a scale of economics, as it shares the same electricity, gases and temperature, which will reduce the costs incurred as opposed to providing a separate incubation unit for the same purpose.

The disclosed device includes the above features in a single device, for the uninterrupted culturing of embryos, which will sustain the maturation, and growth of embryos, from retrieval through to implantation or cryopreservation. The disclosed device may comprise a sealed incubated area, a camera system for imaging and sequencing screens, a vibration system, and an air filtering and recirculating filter system allowing the embryologist to better choose the more viable embryos which will increase the chances of implantation and the likelihood of a live birth.

The disclosed device may provide for the long-term uninterrupted maturation of embryos through to the stage of development desired by the user providing the proper amount of CO², N² and oxygen.

The disclosed device may provide a concise sequence of images of the embryos during development, which will allow the embryologist to better select a viable embryo.

The disclosed device may provide a favorable clear environment by including an effective air filtration system to remove particulates and VOC's from the incubated environment.

The disclosed device may provide an improved device for the long-term uninterrupted maturation of embryos, which contains a real time monitoring system for the key indices such as pH, levels, CO², N², air quality and other key items.

The disclosed device may provide a single device which may be used separately to mature embryos, which will include all the featured needed, such as an incubator section, gas input and the ability to take images at desired times.

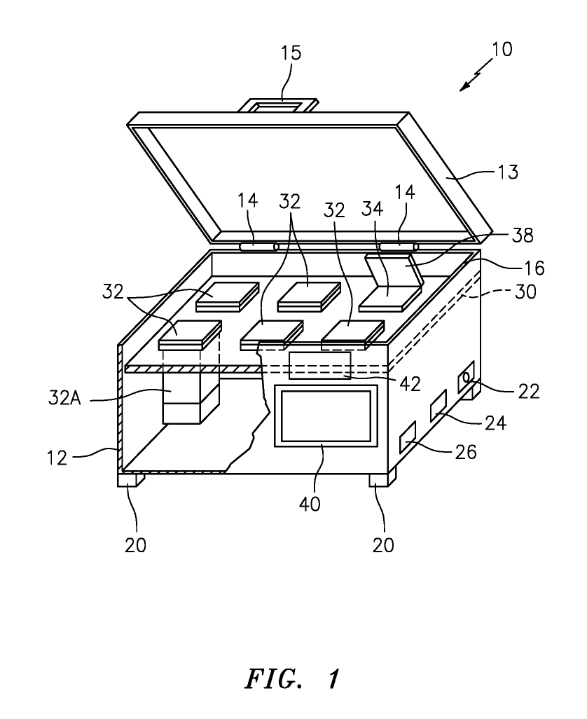

FIG. 1 illustrates the disclosed device 10. The device 10 is a controlled environment for the long-term maturation and imaging observation of embryos. The device 10 has a body 12 with a cover 13, handle on the cover 15, hinges 14 and seal 16 between the body 12 and the cover 13. The exterior body or walls 12 of the device 10 may include insulation to help control the interior temperature of the device 10 and the environment within the device 10. The device 10 contains a heated shelf 30, which is designed to hold the embryonic chambers 32. The shelf 30 may maintain a temperature of about 37° Celsius, holds the embryonic chambers 32 in place, and helps transfer heat to the chambers 32 and separates the upper and lower volumes of the device.

The exterior body or walls 12 of the device 10 may include insulation to help control the interior temperature of the device 10 and the environment within the device 10. The device 10 contains a heated shelf 30, which is designed to hold the embryonic chambers 32. The shelf 30 may maintain a temperature of about 37° Celsius, holds the embryonic chambers 32 in place, and helps transfer heat to the chambers 32 and separates the upper and lower volumes of the device.

The device 10 has incoming gas ports 22 and electrical input connectors 24, and a communication/data port 26. The incoming gas is for the embryonic development, and comprises of CO2, N2 balanced with O2. The incoming gas travels through these ports and then distributed to the chambers. The electrical connections 24 are for providing power to the device 10, and components within the device 10. The communication/data port may be used for the exchange of information of the device 10 such as temperatures, pH readings, gas level reading, air quality readings, images and the like. The input lines for of gas 22 and electrical 24 will be attached to the chambers 32 for gas and electrical needs of the chambers. The chambers 32 will be explained in greater detail below.

In the front portion of the device 10 a display screen 40 is located. The screen 40 may permit the user to set and program information for the chambers 32, timing matters, and may be configured to show images of the embryos in each chamber. The screen 40 may allow the monitoring of each of the chambers 32 and the embryos within (not shown). The screen 40 may be configured to allow users to select the images to be reviewed from these chambers. The screen 40 may present the pertinent information such as temperatures, pH readings, and timing matter to the users. The device may have a security means 42, which in one embodiment may be a security entry system, such as, but not limited to a keypad, or finger print recognition system.

FIG. 1 also shows a cutout 33. Each of the embryonic chambers 32 will have its own cutout 33 that it sits in. The cutout 33 allows the chambers 32 to be viewed from beneath the shelf 30. One of the embryonic chambers 34 is shown with its lid open 38. The device may contain an air purification system (not shown) to purify incoming air and to recirculate air within the device.

The device 10 may have adjustable legs 20, which may allow it to be adjusted, leveled, and may be used for anti-vibration purposes.

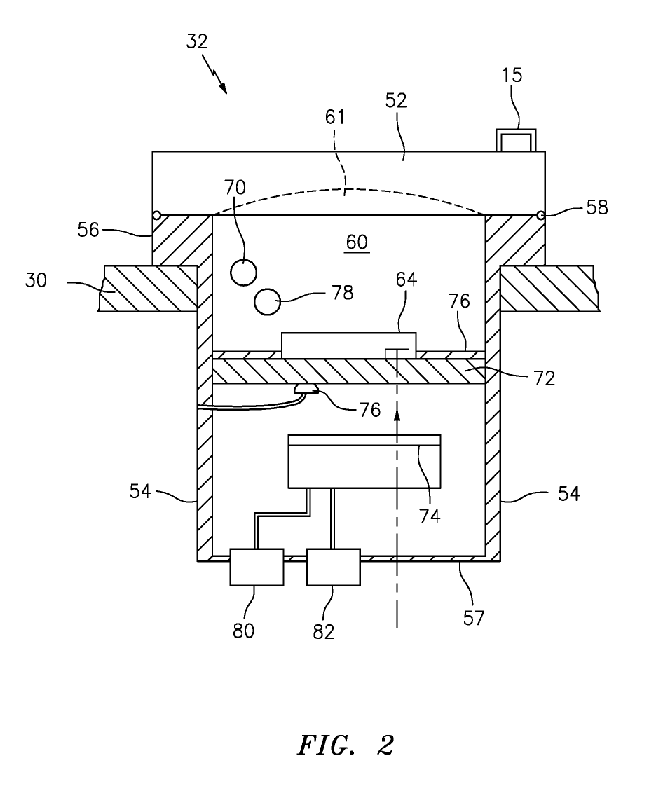

FIG. 2. Shows a cross-sectional view of the chamber 32. The chamber comprises a cover lid 52, which has an airtight seal 58. The chamber comprises four outer walls 54. The walls have an upper ledge 56. The ledge 56 provides a surface where the chamber 32 can hang from the heated shelf 30. This configuration allows the chamber to support a consistent temperature, gas content and environmental conditions when installed in the device 10. The upper section 60 of the chamber 32 is positioned to be adjacent to the plane containing the heated shelf 30. In one embodiment, the upper section 60 may be domed shaped. This configuration of the embryonic chambers will help to maintain a constant temperature, in one example 37° Celsius—the suggested temperature for embryo culture. The chamber 54 walls and the heated shelf 30 may be made of material with good heat transfer properties, such as, but not limited to: stainless steel, aluminum, etc.

The embryos may be held in the incubated volume 60 of the chamber 32, in the specimen dish 64. The chamber 32 may be supplied with a proper mixture of gases, such as but not limited to CO² and N². The gases may be supplied through input ports 70, and a connector port 78. The port 78 may be used to insert various transducers, and other measuring devices such as those for pH level readings. This incubator area 60 has an airtight seal 58 between the lid 52 and the adjacent portion of the chamber 32. The seal 58 keeps impurities out of the chamber 32 and helps maintain the environment within the chamber.

In an embodiment shown in FIG. 2, the interior of the chamber 32 has a heated component 72. In one embodiment, the component 72 may be made out of glass, or any other suitable optically transparent material. The heated component 72 may be maintained at about 37° degrees. Because the component 72 is optically transparent, a camera 74 located in the chamber 32 and below the heated component 72 and the specimen dish 64 can take photographic images of the specimen in the specimen dish 64. The heated component 72 may have a heating system (for heating the component 72 and a temperature sensor 76 for monitoring and maintaining the temperature of the component 72. The temperature sensor 76 may be in communication with a computer for tracking and reporting the temperature. The heated component 72 may be sealed s airtight inside the chamber 32, thereby maintaining the volume above the heated component 72 in a sealed environment, so long as the cover lid 52 is closed. The heated component 72 is used to separate the incubated portion of the chamber from the camera section of the chamber.

In the embodiment shown in FIG. 2, there is a location component 77, placed above the glass 72, to hold the dish 64 in place so that the camera 74 will align the exact focal point of the embryo (not shown) and the camera 74 below. The dishes 64 will have a unique configuration to have the wells of the dish 64 to be aligned with the camera 74. The location component 77 may be a piece of material with a dish-shaped cutout, to locate the dish in the correct location and may have protrusions that line up with recesses in the glass 72.

In the embodiment shown in FIG. 2, the camera 74 and the electronics are below the heated glass 72 and may be in signal communication with connection ports 80 and 82, near or at the bottom 57 of the chamber 32. These connection 80 and 82 may be for electrical connection, gas input connections, sensor connections, camera connections, and any other suitable input, or output.

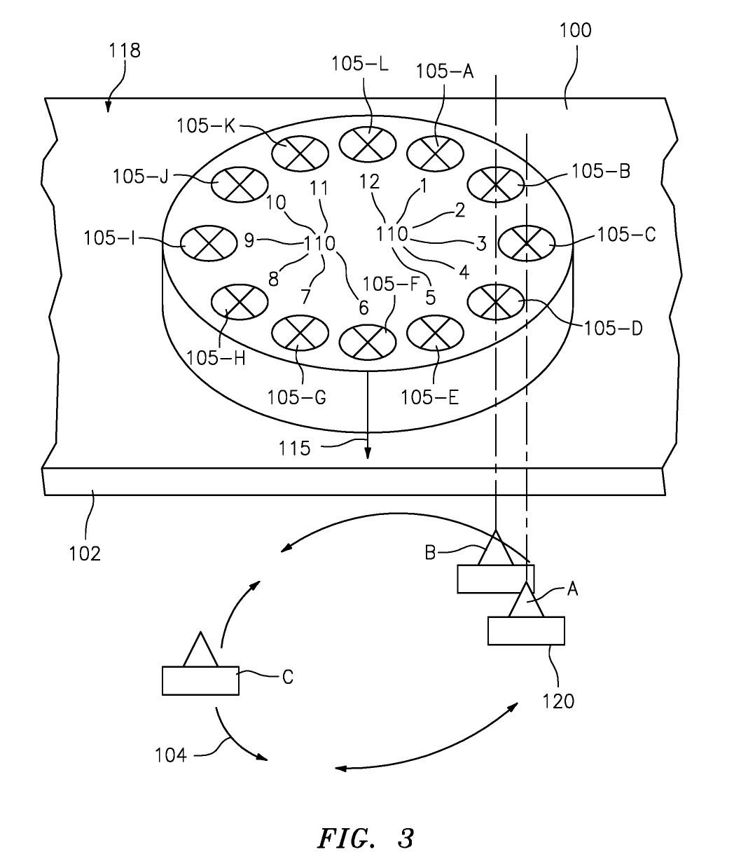

FIG. 3 is a schematic drawing showing a possible relationship of a disclosed culture dish 100 and a disclosed camera system 120. The dish 100 sits on top of the heated glass 102, and is in contact with the glass 102. The heated glass 102 may have an alignment surface 118. The dish 100 may have an alignment component 115. The alignment component 115 may be configured to align the dish 100 with respect to the camera system 120, and may comprise any suitable alignment means, including tongue and groove, alignment member and hole, etc. The glass may be heated to about 37° Celsius. In one embodiment, there may be a single camera 120, which can travel about a generally circular path 104. About the circular path 104 several positions are identified as A, B, and C. The specimen dish 100 may comprise a plurality of wells 105, in this embodiment, there may be 12 wells 105 (105-A through 105-L). The wells may be identified by indices 110. The camera system 120 may rotate either clockwise or counter-clockwise along the path 104. The camera system 120 while moving in its circular path 104, can take images of each of the wells 105 depending on the location of the camera system 120 on the circular path 104. For instance, when the camera system 120 is aligned below well 105-C, when at path position A, then may move counterclockwise take an image of well 105-B at position B. The camera can take images of all at times during the growth, development, or progress of the specimen in the well, or may be programmed to take images only at pre-programmed time intervals. In this figure the camera 120 will take an image of well 105-I when in position C. The camera 120 has the ability to stop under and take images when under any well 105. The images taken can be transmitted to a computer and/or memory located in the disclosed device 10, and can be transmitted to any desired computer location with a proper network connection.

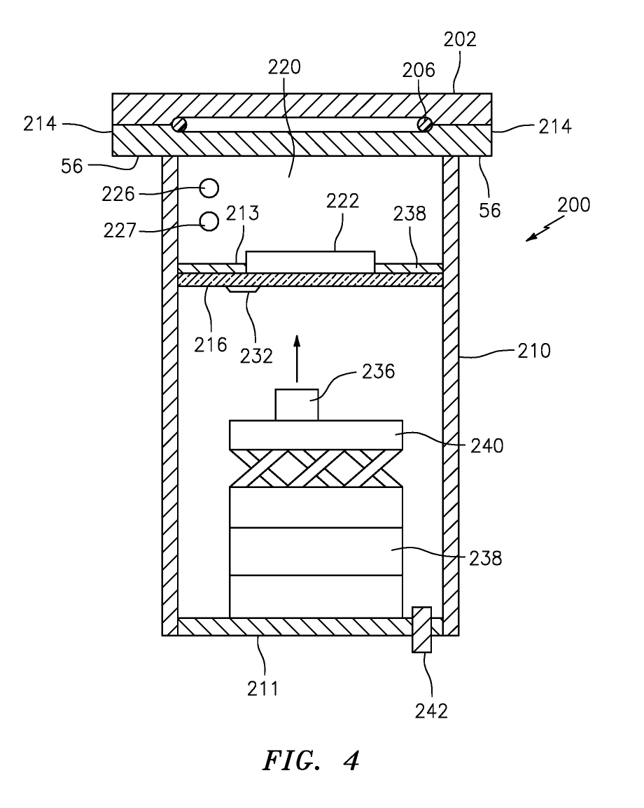

FIG. 4. Shows a cutout view of an additional embodiment of the embryonic chamber 200. The chamber 200 comprises a cover lid 202, which has an airtight seal 206. The chamber 200 comprises at least one outer walls 210. The outer wall 210 has an upper portion 214, which creates an overhang or ledge 56, to support the chamber 200 when it is sitting on and within a heated shelf (not shown). The chamber comprises a bottom 211. The disclosed embodiment enhances the ability for the chamber to support a regulatable temperature, gas content and environmental condition. The upper section 214 of the chamber 200 is configured to be generally in the same plane as the heated shelf 216 and is generally volume 220 of incubation located inside the chamber 200. This configuration will help to maintain a constant temperature in the volume of incubation 220. In one example the temperature in the volume of incubation may be about 37° Celsius, the suggested temperature for embryo culture. The chamber walls 210 may be made of heat transfer materials such as stainless steel, aluminum, or any other suitable heat transferring material. The heated shelf 216 may be made out of any suitable optically transparent material, such as glass.

The embryos may be held in the incubated volume 220 of the chamber 200. A specimen dish 222 is located in the incubated volume 220. The incubated volume 220 will be fed with a proper mixture of gases, CO² and N² through at least one input port 226. There may also be a connector port 228 in the incubated volume 220 for electrical and/or monitoring probes, such as probes for pH level readings. This incubator volume 220 has an airtight seal 204 between the upper section 214 of the chamber 200 and the lid 202, to maintain the gasses and environment in the incubated volume 220.

Still referring to FIG. 4, in this embodiment of the interior of the chamber 220 has a heated shelf 216 component. The heated shelf 216 is heated to maintain about 37 degrees and to allow the camera 236 to take images from below. The heated glass 230 contains both a system for heating the glass, maintaining the temperature, and a sensor 232 for monitoring and maintaining the temperature of the shelf 216, reporting it to the overall heating system and thereby maintaining the required about 37 degrees. The heated glass is airtight sealed in place to help maintain the gasses in the incubated portion 220; the heated shelf 216 is used to separate the incubator portion 220 and the camera 236 portion of the chamber 200. The bottom 211, contains ports 242, which may be ports and connectors for electrical and information input and transfer.

Still referring to FIG. 4, in this embodiment the camera 236 may rest on a camera moving component 238 which will move the camera in two horizontal directions, referred to as the X and Y directions, and also move the camera 236 in a vertical direction, referred to as Z movement. This camera moving component 238 will allow the camera to move below the dish 222 and take precise images at multiple locations of the dish 222, for instance at each well the dish may have (e.g. FIG. 3). The camera moving component 238 will allow the camera 236, to move vertically and horizontally. The camera moving component 238 will also comprise an articulating stage 240. The articulating stage 240 will allow the camera to take images at many angles, which will allow the system to create 3D images of the embryos.

Still referring to FIG. 4, in this embodiment the camera 230 and the electronics are below the heated shelf 216 and may be attached to connections ports 226 and 227, and an electrical port 242 at the bottom 211 of the chamber 200. These connection 226, 227 and 242 may be for electrical connection, gas input connections, sensor connections, camera connections, and others.

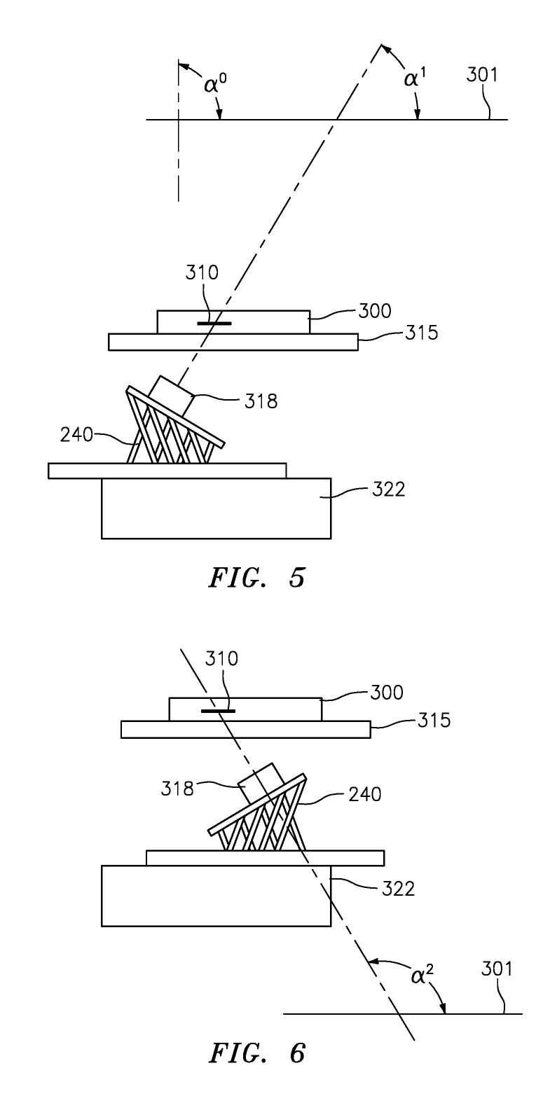

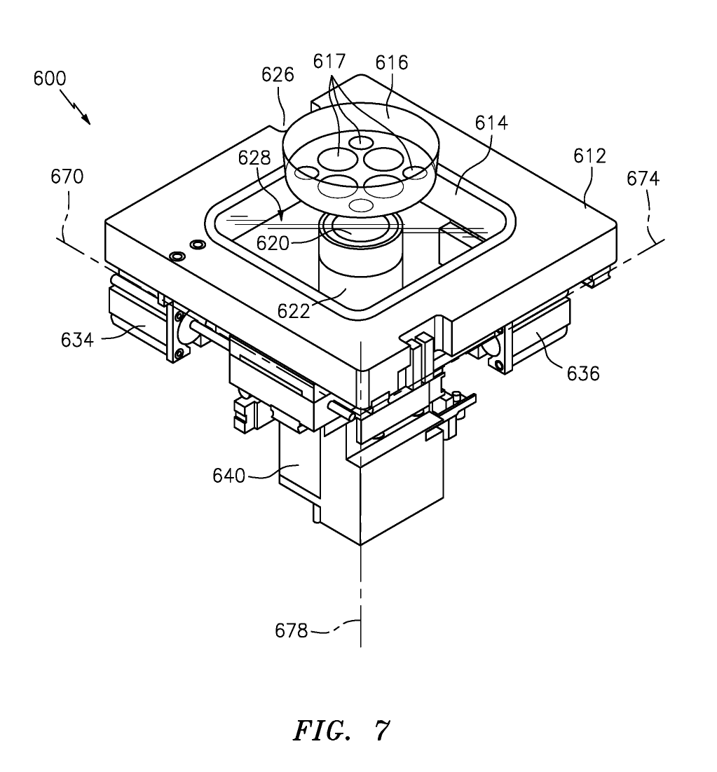

FIGS. 5 and 6 is a schematic view showing how the articulating stage 240 can take various images of the specimen and then to translate the images into 3D images. The embryo 310 sits within a dish 300, on the heated shelf 315. The camera moving component 322 will align the camera 318 in the proper X-Y orientation. The articulating stage 240 will arrange the camera at an angle α¹ with respect to a horizontal plane 301. At angle α¹ the camera 318 can take an angled image of the embryo 310. The articulating stage 240 can change the angle such that, as shown in FIG. 6, the camera angle will be at α². At angle α² the camera 318 can then take an additional image of the embryo 310 at a different angle. The camera 318, in conjunction with the camera moving component 322 and the articulating stage 240 may take as many of these images as required to create a suitable 3D image of the specimen/embryo 310. At angle α⁰ the camera 318 is generally directly aligned with the specimen dish such that angle α⁰ is generally 90° with respect to the horizontal plane 301.

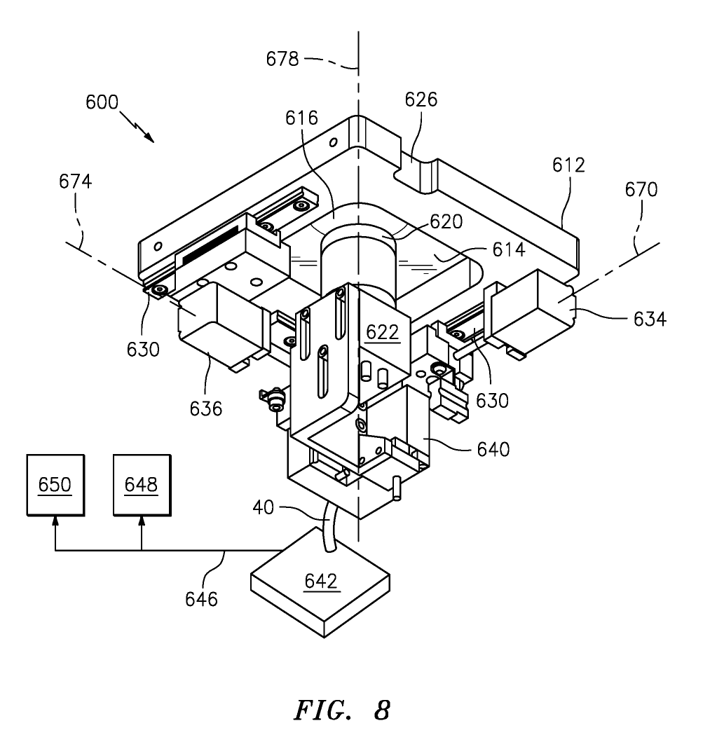

FIG. 7 shows a top perspective view of another embodiment of the disclosed embryonic chamber 600. In this view the chamber walls have been removed for ease of viewing. The chamber comprises a platform 612, and located in the platform is a heated shelf 614. The shelf 614 may be made out of any suitable optically transparent material, such as, but not limited to glass. The platform 612 has an opening so that the platform material, which may be generally opaque, does not obstruct the view from the below the heated shelf 614. Generally coincident with the heated shelf is a view opening 628, which simply allows for a camera located below the platform to have a clear line of sight to the specimen dish 616 through the heated shelf 614. A specimen dish 616 may be removeably attached to the heated shelf 614. The specimen dish 616 may comprise a plurality of wells 617. A the chamber 600 comprises a moveable camera 622 that can be seen through the heated shelf 614. The camera 622 may comprise a camera lens 620. The camera moving component described in previous figures, may comprise a y motor 636, an x motor 634, and a z motor 636. These motors may move the camera 622 in the X-axis 670, Y-axis 674, and Z-axis 678. The platform 612 may have an opening 626, which may be a possible pass through.

FIG. 8 shows the embodiment of the embryonic chamber 600 from FIG. 7, but is a perspective view from the bottom. The embryonic chamber 600 comprises a platform 612. A moveable camera 622 is generally attached to the platform by one or more holding brackets 630. In this embodiment, the x motor 634 may be mounted on one of the holding brackets 626 and the y motor 636 may be mounted on another bracket 626. In this view, one can see a controller 642 is in signal communication with the z motor 640, the controller is also in communication with the x motor 634, and y motor 636. The controller 642 is also in signal communication with a computer 648. The computer 648 may be in communication with a control panel, such as a touch screen control panel 650. The computer 648, and control panel 650 may be part of the controlled environment observation device 10.

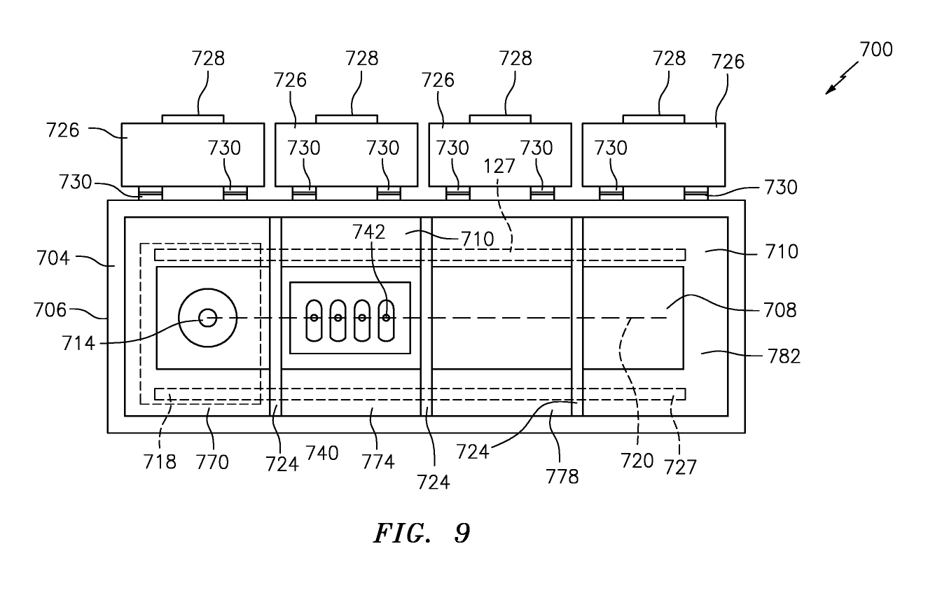

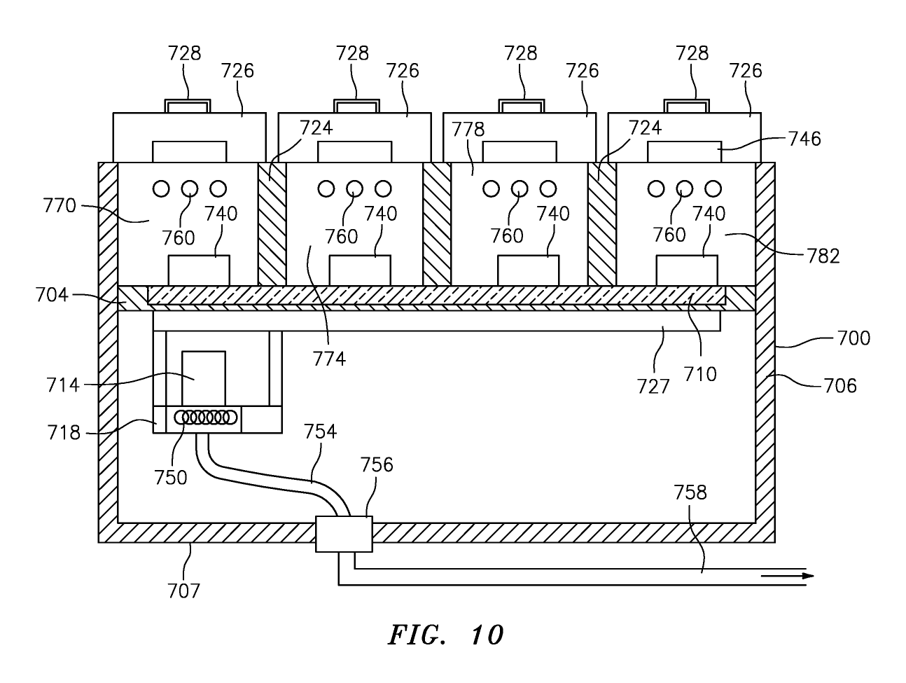

FIG. 9 is a top view of another embodiment of an embryonic chamber 700. This embodiment may be called a multi-area long chamber. In this embodiment the embryonic chamber comprises invention is a series of independent volumes, each of areas, which will hold one or more any specimen dishes. The areas may be divided, so that each volume contains its own environment, or in another embodiment, each volume may share the same or all may be open to one environment. This embodiment uses one camera and an XYZ camera moving system for the imaging of the specimens. Thus, one camera system may be used to view a plurality of specimen dishes in a plurality of volumes. Shown in FIG. 9 are the platform 704, and chamber walls 706. Heated shelf 710 is supported by the platform 704. The platform 704 has an opening 708 where the shelf 710 sits, and allows for the camera 714 to view the specimen dishes 740 and embryos 742 in the dishes 740 from below. The chamber walls 706 and divider walls 724 generally define four volumes 770, 774, 778, 782. In other embodiments, less or more volumes may be provided in the embryonic chamber 700, depending on the needs of the end user. A specimen dish 740 is shown on a heated shelf 710. A camera 714 can be seen under the heated shelf 110. The camera 714 is in operable communication with an XYZ camera moving system 718. In this embodiment, the camera 714 can generally move about camera movement path 720. The movement path 720 may generally be on the X axis of the XYZ camera moving system 718, and fine movements may be made on the Y axis and Z axis to center the specimen in the camera focal point. The embryonic chamber may comprise a cover 726 for each of the volumes 804, 808, 812, 816. Each cover 726 may have a handle 728. Each of the covers 726 may be attached to the platform or chamber walls by hinges 730. A support bracket 727 is also shown.

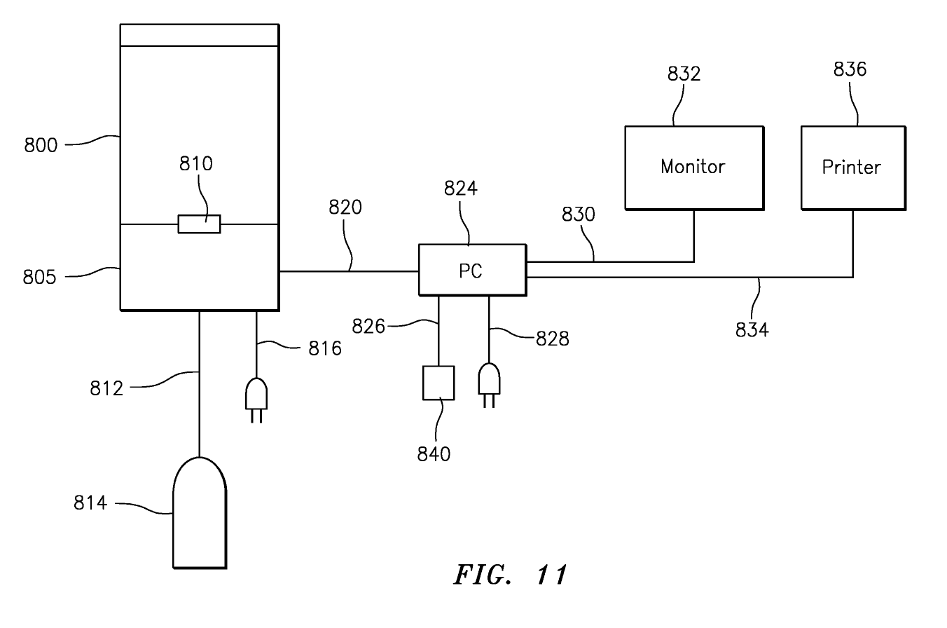

FIG. 10 is a side view of the embryonic chamber 700 shown in FIG. 9. In this view, the support bracket 727 is shown attached to the xyz camera moving system 718. The camera 714 is below the heated shelf 710 and can take photographs and other images up in the specimen dishes 740 through the heated shelf 710 (which is generally transparent). The xyz camera moving system 718 comprises motors to move the camera 714 about X, Y, and Z axes. The motors are not shown in this view. The xyz camera moving system 718 also comprises a controller 750, which is in communication with the motors, and in communication with a communication cable 754. The communication cable 754 will be in communication with a computer located on the controlled environment observation device 10. The computer (not shown) may be in communication with a touch screen control panel (also not shown). The cable 754 may be plugged into a connector 756 located in the chamber wall 706 or chamber floor 707. The connector 756 may be in communication with another cable 758 that is in communication with the computer.

FIG. 11 shows another embodiment of the disclosed device. In this example of the embodiment a single chamber 800 sits in a landing dock 805. A connector 810 connects the chamber 800 to the landing dock 805. The landing dock 805 is in communication with one or more gas sources 814, which supply the dock 805 with the correct balances of gases through the tubing 812. The landing dock 805 is powered thru electrical connector 816. The landing dock 805 is connected by cables 820 to a computer 824, which is powered through power connector 828 and is connected to the Internet and or other networks 840. The computer 824 is in communication to a monitor 832, and a printer 836 via a cable 836 or alternatively through a wireless network. This embodiment allows the chamber 800 to be used independent of a larger containment system, not shown, and would allow the user to remove the chamber form the larger unit and maintain the controlled environment of gases and temperature and to be used in a separate area of the laboratory or workstation, for observation purposes or to be held for additional procedures, such as fertilization, ICSI, PGD or other testing protocols.

Throughout this patent application, numerous references will be made regarding computers, servers, services, engines, modules, interfaces, portals, platforms, or other systems formed from computing devices. It should be appreciated that the use of such terms are deemed to represent one or more computing devices having at least one processor configured to or programmed to execute software instructions stored on a computer readable tangible, non-transitory medium. For example, a server can include one or more computers operating as a web server, database server, or other type of computer server in a manner to fulfill described roles, responsibilities, or functions.

It should be noted that the terms “first”, “second”, and “third”, and the like may be used herein to modify elements performing similar and/or analogous functions. These modifiers do not imply a spatial, sequential, or hierarchical order to the modified elements unless specifically stated.

While the disclosure has been described with reference to several embodiments, it will be understood by those skilled in the art that various changes may be made and equivalents may be substituted for elements thereof without departing from the scope of the disclosure. In addition, many modifications may be made to adapt a particular situation or material to the teachings of the disclosure without departing from the essential scope thereof. Therefore, it is intended that the disclosure not be limited to the particular embodiments disclosed as the best mode contemplated for carrying out this disclosure, but that the disclosure will include all embodiments falling within the scope of the appended claims.

What is claimed is:

1. A controlled environment observation device comprising:
a body;
a heated shelf located in the body;
a plurality of shelf openings in the heated shelf;
a plurality of embryonic chambers removably installed in the plurality of shelf openings, each of the plurality of embryonic chambers comprising:
an optically transparent heated shelf;
a specimen dish removably attached to the top of the optically transparent heated shelf, the specimen dish having a generally optically transparent bottom;
an x,y,z camera system located in each of the embryonic chambers and below the specimen dish and the optically transparent heated shelf;
a computer located in the controlled environment observation device; and
a controller in signal communication with the xyz camera system and the computer.

2. The controlled environment observation device of claim 1, wherein each of the embryonic chambers can be individually removed and replaced for service or maintenance while the remaining embryonic chambers remain operational.

3. The controlled environment observation device of claim 1, wherein each plurality of embryonic chambers has an upper ledge configured to allow the embryonic chamber to hang from the heated shelf when in the shelf opening.

4. The controlled environment observation device claim 1, wherein each x, y, z camera system also comprises an articulating stage that allows a camera in the x, y, z camera system to change its angle with respect to the specimen dish.

5. The controlled environment observation device claim 4, wherein each articulating stage can change the camera angle α0, to angles α1 and α2, where α0 is generally 90° with respect to a horizontal plane.

6. The controlled environment observation device claim 1, wherein each specimen dish comprises at least one well, wherein the well is a depression in the dish, that is configured to allow a biological specimen in the specimen dish to sink into and center in the well.

7. The controlled environment observation device claim 6, wherein each well has generally an inverted truncated cone shape.

8. The controlled environment observation device claim 1, where each embryonic chamber has a controlled incubated environment configured for the proper growth and development of a biological specimen in the specimen dish.

9. The controlled environment observation device of claim 8, further comprising:
a gas inlet located in the controlled incubated environment, the gas inlet in fluid communication with a gas supply.

10. The controlled environment observation device claim 8, where each controlled incubated environment contains transducers to measure environmental conditions in the controlled incubated environment, and where the transducers are in signal communication with the computer.

11. The controlled environment observation device claim 10, where the transducers comprise:
a pH measuring transducer;
a temperature measuring transducer;
an air composition transducer.

12. The controlled environment observation device claim 8, where each embryonic chamber has a sealable chamber that encloses the incubated environment.

13. The controlled environment observation device claim 12, where each sealable chamber comprises at least one wall, a top, and a bottom.

14. The controlled environment observation device of claim 1, further comprising:
a touch screen control panel located on the body and in signal communication with the computer.

15. The controlled environment observation device of claim 1, wherein the computer contains a security program that prevents unauthorized access to the device.

16. The controlled environment observation device of claim 1 further comprising a joystick control in communication with the computer, and configured to control the movement and location of a camera in the xyz camera system.

17. The controlled environment observation device of claim 1, wherein each plurality of embryonic chambers has at least one gas port.

18. The controlled environment observation device of claim 1, wherein each x, y, z camera system comprises:
an x motor in operable communication with a camera;
a y motor in operable communication with the camera, and where the x motor and y motor can move the camera generally about a horizontal plane below the specimen dish;
a z motor in operable communication with the camera, and where the z motor can move the camera generally towards and way from the specimen dish.

19. The controlled environment observation device of claim 1, wherein each of the embryonic chambers further comprises:
a platform;
an opening in the platform housing the optically transparent heated shelf.

20. A controlled environment observation device comprising:
a body;
at least one multi-area embryonic chamber located in the body, the multi-area embryonic chamber comprising:
at least one outer wall forming a perimeter;
a platform generally located within the perimeter;
an opening in the platform;
an optically transparent heated shelf located in the opening;
a first plurality of inner walls forming a second plurality of volumes with the at least one outer wall;
a second plurality of specimen dishes configured to be removeably attached to the optically transparent heated shelf in each of the second plurality of volumes, each of the specimen dishes having a generally optically transparent bottom;
an x,y,z camera system located beneath the optically transparent heated shelf, and configured to move a camera to any of the specimen dishes.
